(19) United States
(12) Patent Application Publication
(10) Pub. No.: US 2009/0074132 A1
(43) Pub. Date: Mar. 19, 2009

(54) COMPUTER TOMOGRAPHY APPARATUS AND METHOD OF EXAMINING AN OBJECT OF INTEREST WITH A COMPUTER TOMOGRAPHY APPARATUS

(75) Inventors: Jens-Peter Schlomka, Hamburg (DE); Udo Van Stevendaal, Ahrensburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/577,738

(22) PCT Filed: Oct. 24, 2005
(86) PCT No.: PCT/IB05/64575

§ 371 Date: Apr. 23, 2007

(30) Foreign Application Priority Data
Oct. 26, 2004 (GB) 0423707.9

(51) Int. Cl. A61B 6/00

(52) U.S. Cl. 378/19

(57) ABSTRACT

A computer tomography apparatus (300) for examination of an object of interest, the computer tomography apparatus (300) comprising an X-ray source (302) adapted to emit X-rays to an object of interest, first detecting elements (304) adapted to detect X-rays coherently scattered from an object of interest (301) in an energy-resolving manner, second detecting elements (305) adapted to detect X-rays coherently scattered from an object of interest (301) in a non-energy-resolving manner, and a determination unit (306) adapted to determine, based on detecting signals received from the first detecting elements (304) and/or from the second detecting elements (305), structural information concerning the object of interest (301).

The invention relates to the field of X-ray imaging. In particular, the invention relates to a computer tomography apparatus and to a method of examining an object of interest with a computer tomography apparatus.

Over the past several years, X-ray baggage inspections have evolved from simple X-ray imaging systems that were completely dependent on an interaction by an operator to more sophisticated automatic systems that can automatically recognize certain types of materials and trigger an alarm in the presence of dangerous materials. An inspection system has employed an X-ray radiation source for emitting X-rays which are transmitted through or scattered from the examined package to a detector.

An imaging technique based on coherently scattered X-ray photons is the so-called “coherent scatter computer tomography” (CSCT) technique. CSCT is a technique that produces images of the low angle scatter properties of an object of interest. These depend on the molecular structure of the object, making it possible to produce material-specific maps of each component. The dominant component of low angle scatter is coherent scatter. Since coherent scatter spectra depend on the atomic arrangement of the scattering sample, coherent scatter computer tomography (CSCT) is a sensitive technique for imaging spatial variations in the molecular structure of baggage or biological tissue across a two-dimensional object section.

By using a fan-shaped primary beam combined with two-dimensional detectors, transmission tomography and scatter tomography can be measured simultaneously.

A conventional cone-beam computer tomography (CT) scanner is equipped with a non-energy resolving two-dimensional detector, whereas energy-resolved CSCT requires a one-dimensional or two-dimensional energy resolving detector. Therefore, in a cone-beam CT/CSCT scanner, the two techniques can be measured simultaneously or subsequently: non-energy resolving CSCT using the CT detector, and energy resolving CSCT using the extra energy-resolving detector.

DE 10009285 A1 and U.S. Pat. No. 6,470,067 B1 disclose the principle of an imaging method based on coherently scattered X-ray radiation. For this purpose, a small fan-beam of radiation with a small divergence out of the fan-plane is guided to the object. Then, the transmitted radiation is measured as well as the radiation which is scattered by the object out of the plane of the fan.

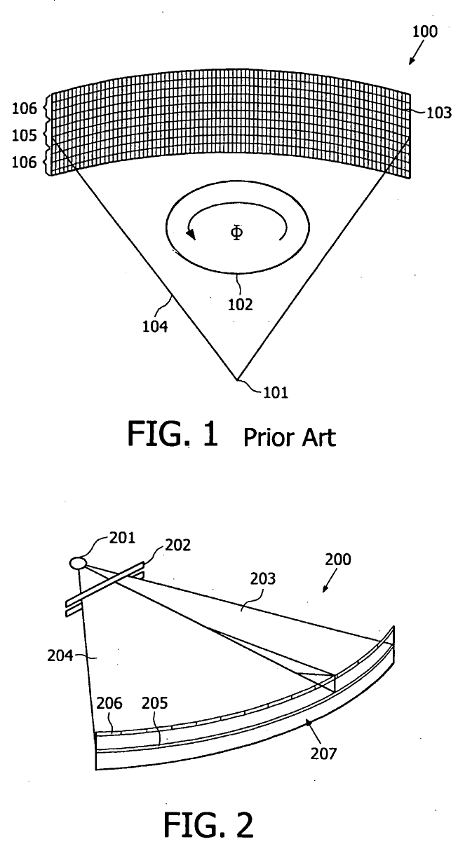

The geometry of such a computer tomography apparatus 100 known from the prior art, is shown in FIG. 1.

FIG. 1 shows a CSCT computer tomography apparatus 100 for examination of an object of interest 102, the CSCT computer tomography apparatus 100 having an X-ray source 101 which produces a fan beam 104 which impinges the object of interest 102. Coherently scattered radiation scattered by the object of interest 102 impinges a two-dimensional detector 103 which has a centred detector line 105 and decentred detector lines 106. The central detector line 105 measures transmitted radiation of the primary fan beam 104. The decentred detector lines 106 measure scattered radiation. However, the detector 105, 106 has no or only a very bad (ΔE/E>20%) energy resolution.

However, for many applications, it would be advantageous to have a computer tomography apparatus having an improved resolution.

The invention intends to allow investigating an object of interest with an improved resolution.

According to the invention a computer tomography apparatus and a method of examining an object of interest with a computer tomography apparatus with the features according to the independent claims are provided.

The computer tomography apparatus for examination of an object of interest comprises an X-ray source adapted to emit X-rays to an object of interest, first detecting means adapted to detect X-rays coherently scattered from an object of interest in an energy-resolving manner, second detecting elements adapted to detect X-rays coherently scattered from an object of interest in a non-energy-resolving manner, and a determination unit adapted to determine, based on detecting signals received from the first detecting elements and/or from the second detecting elements, structural information concerning the object of interest.

Further, a method of examining an object of interest with a computer tomography apparatus is provided, comprising the steps of emitting X-rays to an object of interest, detecting X-rays coherently scattered from the object of interest in an energy-resolving manner, detecting X-rays coherently scattered from the object of interest in a non-energy resolving manner, and determining, based on energy-resolved detecting signals and/or on non-energy-resolved detecting signals, structural information concerning the object of interest.

The characteristic features according to the invention—particularly the simultaneous measurement of coherently scattered X-rays in an energy-resolving manner and in a non-energy resolving manner within one and the same apparatus with the opportunity to evaluate the data in a combined manner—provide complementary information concerning the object of interest and thus allow a highly accurate analysis of the properties of the object. The invention combines the advantages of energy-resolved coherently scattering of X-rays (e.g. high resolution and detailed structural information) with the advantages of non-energy-resolved coherently scattering of X-rays (e.g. fast and easy analysis). By acquiring all these signals, a high quality analysis of an object of interest is possible. Particularly, the energy-resolved detection improves the spectral resolution of the system. On the other hand, the computer tomography apparatus measures non-energy resolving detection signals allowing a fast acquisition and evaluation of the data. The invention allows for any particular application of the computer tomography apparatus to consider frame conditions of such an application with a high degree of flexibility, since, depending on a desired degree of resolution and depending on a desired time resolution, either the detecting signals measured in an energy-resolving manner or the detection signals measured in a non-energy-resolving manner, or both of these signal groups can be used for estimating structural properties of the object of interest. Thus, the invention combines energy-resolved CSCT and non-energy-resolved CSCT. The invention may further combine conventional CT with CSCT in a single apparatus.

According to the invention, the add-on character of CSCT for a cone-beam CT scanner is emphasized, since the invention uses an anyway-present CT detector in an efficient way. It maximizes the flexibility of the setup and uses as many possible photons as needed, thus increasing speed and detection rate. The invention can further be implemented in the frame of a combined cone-beam CT/fan-beam CSCT scanner. Particularly, the invention can be advantageously applied in the field of CSCT baggage inspection.

In contrast to DE 10009285 A1, the invention is not restricted to use only non-energy-resolving detectors, but additionally implements energy-resolving detectors.

Further, the invention is not restricted to use only energy-resolving detectors for scatter measurements, but additionally uses a non energy-resolving 2D detector. In the configuration shown in FIG. 2, the non-energy-resolving detector is solely used for measuring the transmitted radiation, which results in a conventional CT image.

A pure energy-resolving detection computer tomography apparatus, as the one shown in FIG. 2, is restricted to include a plurality of energy-resolving detector rows, however, does not allow the simple and efficient non-energy-resolving detection signals to be captured and provided for a subsequent analysis.

The invention combines the good spectral resolution of energy-resolved CSCT, even when using polychromatic primary radiation, with the proper time resolution of non-energy-resolved CSCT. In other words, the invention combines the two techniques of energy-resolved CSCT and non-energy-resolved CSCT. This yields profit in the light of the fact that current CT scanners are cone-beam CT scanners. Such a scanner can be used, after a fan-beam collimation, for non-energy-resolved CSCT. The additional energy-resolving detector can be used for energy-resolved CSCT.

The invention describes a geometry for a combined measurement and method for evaluation of the captured data.

It is a particular advantage of the invention that non-energy-resolved CSCT and energy-resolved CSCT are combined, since a cone-beam CT scanner anyway includes a two-dimensional detector, so that non-energy-resolved CSCT can be measured without providing any additional hardware.

An important measure for X-ray scatter investigations is the so-called momentum transfer parameter x (which is equivalent to the wave-vector transfer multiplied with a fixed factor). The momentum transfer parameter can be extracted by measuring the scatter angle and the energy of a scattered photon applying

$$x = \frac{E}{hc} sin ( Θ / 2 ) \qquad ( 1 )$$

with E the energy of the photon, h Planck's constant, c the speed of light and the scatter angle.

A ‘non-energy resolved’ scatter measurement does not allow determination of the energy of the scattered photon with an accuracy of better than 20%. In such a measurement, the parameter x is calculated by using the average energy of the incoming radiation as ‘E’ and determining the scatter angle. If more than one value for x has to measured, more than one detector has to be used to measure several scatter angles.

An ‘energy resolved’ scatter measurement allows determination of the energy of the scattered photon with an accuracy of better than 20%. Here, a range of values for the parameter x can be measured for each individual scatter angle Θ, if a polychromatic X-ray source is used. A polychromatic source provides photons in a wide range of energies (e.g. 50-150 keV). Thus, one energy-resolving detector can be sufficient to cover a range of values of x.

Referring to the dependent claims, further preferred embodiments of the invention will be described in the following.

Next, preferred embodiments of the computer tomography apparatus will be described. These embodiments may also be applied to the method of examining an object of interest with a computer tomography apparatus.

The computer tomography apparatus may be adapted as a coherent scatter computer tomography apparatus (CSCT), i.e. the computer tomography apparatus may be configured and operated according to the CSCT technology described above.

A collimator may be arranged between the X-ray source and the first and the second detecting elements, the collimator being adapted to collimate an X-ray beam emitted by the X-ray source to form a fan-beam. A fan-beam is the preferred beam-shape of the CSCT technology. Implementing such a collimator preferably having an elongated slit, it is possible to use almost any desired X-ray source, since a properly shaped collimator produces a fan-beam from any type of primary X-ray beam geometry.

The first detecting elements and the second detecting elements may form a two-dimensional detector array. Thus, all of the detecting elements can be provided within one and the same detector device, which can be, for example, a semiconductor detector or a scintillation counter.

A distance between the X-ray source and the first detecting elements may be essentially similar to the distance between the X-ray source and the second detecting elements. Such a configuration may simplify the evaluation of the signals detected by the two different detector elements, since differences in the detected signals due to different distances between detector and radiation beam are avoided or suppressed.

Alternatively, a distance between the X-ray source and the first detecting elements may differ from a distance between the X-ray source and the second detecting elements. This configuration may be advantageous, since there are scenarios, in which the increase of the distance between the X-ray source/the object on the one hand and detecting elements on the other hand may improve the resolution of different scattered X-ray beams which have a similar scatter angle, since increasing the distance may allow to distinguish two different resonances which may overlap when a too small distance is selected. By flexibly allowing, if desired, to increase the distance between the X-ray source/the object on the one hand and detecting elements on the other hand, the resolution of the measurement can be improved.

The first detecting elements may be adapted to detect X-rays scattered from an object of interest into a first angle portion, and the second detecting elements may be adapted to detect X-rays scattered from an object of interest in a second angle portion. Particularly, the first angle portion may cover larger angles than the second angle portion. According to this configuration, the non-energy-resolved detecting signals are detected at lower scatter angles than the energy-resolved detecting signals which are detected at larger angles. This is advantageous, since meaningful energy-resolved detection signals may preferably appear at relatively large angles.

The first detecting means may be divided into first sub-elements and into second sub-elements, wherein the second detecting elements may be arranged between the first sub-elements and the second sub-elements. Thus, a sandwich-like structure of the detecting means is achieved, having a central portion with the second detecting elements related to the non-energy-resolved CSCT, and at both sides of this central portion, sub-elements related to the first detecting elements and the energy-resolved CSCT may be arranged. Thus, a very compact configuration is achieved.

The computer tomography apparatus may further comprise one or more collimating blades arranged on (preferably attached to) the first detecting elements and/or arranged on (preferably attached to) the second detecting elements and being aligned to point towards the X-ray source. By providing such blades from an X-ray absorbing material (e.g. made of Lead or Tungsten material), the meaningfulness of the detected signals can be improved, since undesired background radiation which is not related to small scatter angles can be eliminated, so the useful signal is more pronounced compared to the background.

The first detecting elements and the second detecting elements may be provided with a common casing. This allows a very compact configuration of the apparatus.

Alternatively, the first detecting elements may be provided in a first casing and the second detecting elements may be provided in a second casing which is different from the first casing. According to this configuration, a retrofitting of an existing apparatus in order to implement the system of the invention is possible, so that the invention can be later integrated in an existing device.

The X-ray source may be arranged with respect to the first detecting elements and the second detecting elements such that X-rays being transmitted from an object of interest impinge on a non-central portion of the first detecting elements and/or of the second detecting elements. In such an asymmetric configuration, a shift of the detectors, the X-ray source or of blades of a collimating means can be carried out, increasing the sensitivity. In such a set-up the non-energy-resolving detector and the energy-resolving detector may cover approximately the same range of scatter angles. The first detector measures scatter to one side of the primary fan and the latter detector measures the scatter on the other side of the fan.

Further, the computer tomography apparatus may be adapted such that the first detecting elements and the second detecting elements are arranged on both sides of a primary fan-beam as emitted X-rays such that a measured angular range of the first detecting elements and the second detecting elements are essentially equal.

The X-ray tomography apparatus according to the invention may be configured as one of the group consisting of a baggage inspection apparatus, a medical application apparatus, a material testing apparatus and a material science analysis apparatus. However, the most preferred field of application of the invention is baggage inspection, since the refined functionality of the invention allows a secure and reliable analysis of the content of a baggage item allowing detection of suspicious content, even enabling determination of the type of material inside such a baggage item. The invention creates a high-quality automatic system that can automatically recognize certain types of materials and, if desired, trigger an alarm in the presence of dangerous materials. Such an inspection system has-employed the computer tomography apparatus of the invention with an X-ray radiation source for emitting X-rays which are transmitted through or scattered from the examined package to a detector, allowing to detect coherently scattered radiation in an energy-resolved manner and in a non-energy-resolved manner.

In the following, preferred embodiments of the method of examining an object of interest with a computer tomography apparatus will be described. However, these embodiments also apply for the computer tomography apparatus of the invention.

The method may further comprise the steps of detecting X-rays transmitted through the object of interest, and determining, based on the detected transmitted X-rays, whether a further analysis is necessary.

The method of the invention may further comprise the steps of determining, based on non-energy-resolved detecting signals without considering energy-resolved detecting signals, structural properties concerning the object of interest. Further, the determined structural properties may be analyzed, to decide whether a further examination is desired or not. Only if it is decided that a further determination is desired, it may be determined, based on energy-resolved detecting signals, structural properties concerning the object of interest. According to this embodiment, a more complex and time-consuming energy-resolved determination can be avoided in cases in which the more simple and fast non-energy-resolved determination is already sufficient to yield a result with sufficient accuracy. Only in cases in which a detailed analysis is necessary, the signals according to the energy-resolved detection are taken into account to improve and to refine the meaningfulness of the structural properties estimated by the examination method of the invention.

For instance, a baggage which is, after having performed a non-energy-resolved analysis, considered to be suspicious can be inspected, if desired, subsequently in more detail by including also an energy-resolved analysis.

According to the method of the invention, additional energy-resolved scatter data may acquired in a subsequent step after acquiring non-energy-resolved scatter data.

According to the described embodiment, the decision whether a further determination is desired may be taken including comparing structural properties concerning the object of interest determined based on non-energy-resolved detecting signals with data of a database (e.g. an electronic library). By such a comparison with pre-known data concerning an expected structure of the object, it can be determined with good reliability whether an additional investigation is necessary.

According to another embodiment, structural properties concerning the object of interest may be determined based on energy-resolved detecting signals, wherein the non-energy-resolved detecting signals may be used as a convergence criterion during reconstruction. In other words, when analyzing the more complicated energy-resolved detection signals, more general information (“frame conditions” according to a model) from the non-energy-resolved detecting signals may be included in the evaluation so that it is avoided that a fit of data is trapped in a wrong minimum, i.e. an artefact which does not reflect the real physical situation.

Embodiments of the present invention will not be described, by way of example only, and with reference to the accompanying drawings wherein:

FIG. 1 shows a computer tomography apparatus according to the prior art,

FIG. 2 shows a computer tomography apparatus including energy-resolved CSCT,

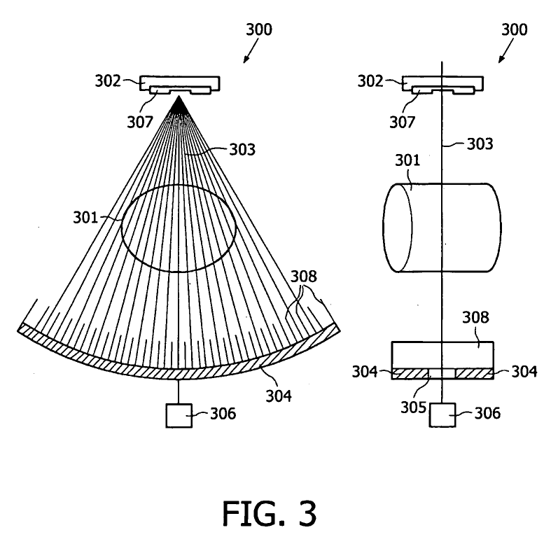

FIG. 3 shows a computer tomography apparatus according to a first embodiment of the invention,

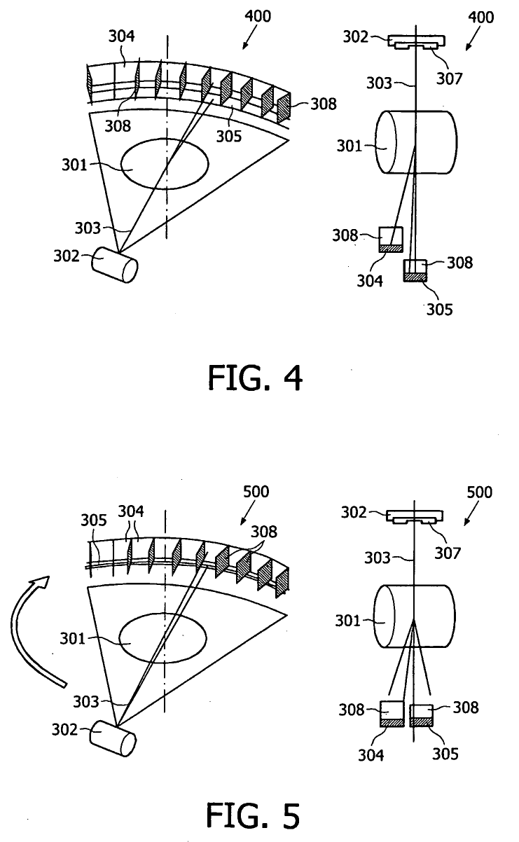

FIG. 4 shows a computer tomography apparatus according to a second embodiment of the invention,

FIG. 5 shows a computer tomography apparatus according to a third embodiment of the invention,

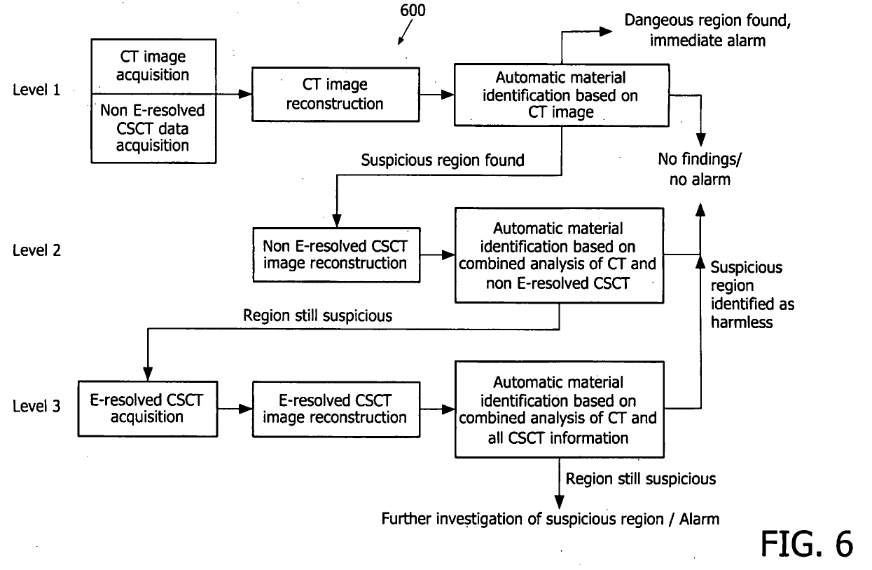

FIG. 6 shows a flow diagram illustrating a method of examining an object of interest with a computer tomography apparatus according to an embodiment of the invention.

The illustration in the drawings is schematically. In different drawings, similar or identical elements are provided with the same reference signs.

In the following, referring to FIG. 2, a computer tomography apparatus 200 will be described having implemented energy-resolved CSCT.

The CSCT computer tomography apparatus 200 has an X-ray source 201 for emitting an X-ray beam which is guided through a slit collimator 202 to form a primary fan-beam 203 impinging an object to be located in an object region 204. A multi-line detector 207 is constituted by a central detection element 205 (i.e. a central row for the detection of X-rays of the fan-beam transmitted through an object) and by energy-resolving detection elements 206 (i.e. energy-resolving detector lines).

Thus, FIG. 2 shows a geometry for pure energy-resolved CSCT. The central detection line 205 measures transmitted radiation, whereas the one or more detection lines 206 are configured to perform an energy-resolving measurement. However, with the apparatus 200 shown in FIG. 2, a complicated analysis of the signals is necessary in all cases, since the apparatus 200 is based merely on complex energy-resolving.

According to the invention, energy-resolving evaluation is combined with non-energy-resolving evaluation, to have both, a high performance and the opportunity of a simple evaluation.

In the following, referring to FIG. 3, a CSCT computer tomography apparatus 300 according to a first embodiment of the invention will be described in detail.

FIG. 3 shows a plan view and a cross-sectional view of the CSCT (coherent scatter computer tomography) computer tomography apparatus 300.

The CSCT computer tomography apparatus 300 is adapted for examination of an object of interest 301, for example a baggage item to be inspected in the frame of a baggage inspection system. An X-ray tube 302 emits a primary fan-beam 303 as X-rays to the object of interest 301. First detecting pixels 304 are adapted to detect X-rays coherently scattered from the object of interest 301 in an energy-resolving manner. Second detecting pixels 305 are adapted to detect X-rays coherently scattered from the object of interest 301 in a non-energy-resolving manner. Further, a microprocessor 306 as a determination unit is adapted to determine, based on detecting signals received from the first detecting pixels 304 and/or from the second detecting pixels 305, structural and material properties concerning the object of interest 301, i.e. allow to analyze the content of the piece of baggage under examination. For the analysis, the microprocessor 306 may use, in a user-defined or in an automatic manner, either only the non-energy-resolved signals, or only the energy-resolved signals, or both the energy-resolved signals and the non-energy-resolved signals. The analysis may include tomographic reconstruction of the scatter signals from different viewing angles. As can be seen from FIG. 3, the first detecting pixels 304 and the second detecting pixels 305 form a two-dimensional detector array.

Further, a collimator 307 is arranged between the X-ray tube 302 and the first and the second detecting elements 304, 305, the collimator 307 being adapted to collimate an X-ray beam emitted from the X-ray tube 301 to form the primary fan-beam 303. A distance between the X-ray tube 302 and the first detecting pixels 304 essentially equals a distance between the X-ray tube 302 and the second detecting elements 305. Due to the geometrical configuration of the detecting elements 304, 305, the first detecting elements 304 are adapted to detect X-rays scattered from the object of interest 301 into a first angle portion, and the second detecting elements 305 are adapted to detect X-rays scattered from the object of interest 301 into a second angle portion. The second detecting pixels 305 are provided between different portions of the first detecting pixels 304. Further, a plurality of collimating blades 308 made of lead are arranged attached to the first detecting elements 304 and attached to the second detecting elements 305 and are aligned to point towards the X-ray tube 301. According to this configuration, the percentage of meaningful radiation impinging the detector elements 304, 305 is increased, since background radiation deteriorating the sensitivity of the detector is efficiently suppressed.

Thus, FIG. 3 shows an advantageous geometry for the computer tomography apparatus of the invention. The energy-resolving detector 304 is located essentially at the same distance from the X-ray beam like the non-energy-resolving detector 305, and both detector elements are located behind a focusing blade or aperture, namely behind the collimating blades 308. Both detectors 304, 305 are provided in one and the same casing allowing a common design.

The primary beam emitted from the X-ray tube 302 is collimated to form the primary fan-beam 303, i.e. only one or few lines of the CT detector 304, 305 are directly impinged by the transmitted radiation. Such detector lines which are arranged more remotely from the primary radiation already detect scattered radiation. This scattered radiation can be reconstructed according to known methods, and thus, a non-energy-resolving CSCT image is obtained using signals detected by the second detector elements 305. At even larger scattering angles, the energy-resolving detector 304 is arranged. The radiation detected here is reconstructed under consideration of the different scattering angles, and thus an energy-resolved CSCT pixel image is obtained, i.e. the measured “coherent scatter form factor” has a better resolution concerning the wave vector transfer.

Thus, FIG. 3 shows a geometry for a common/combined measurement of energy-resolved and of non-energy-resolved CSCT. In front of both detectors 304, 305, the collimator blades 308 are arranged which are focused to be aligned in direction towards the X-ray source 302.

FIG. 3 shows an embodiment in which the non-energy-resolving detector 304 is located around the fan-beam area 303 and may detect direct radiation as well as scattered radiation. A second energy resolving detector 304 is located beside of the first one 305. The collimator 308 focused to the X-ray source 302 is placed above both detectors 304, 305.

In the following, referring to FIG. 4, a CSCT computer tomography apparatus 400 according to a second embodiment of the invention is described. FIG. 4 shows a schematic perspective view of the CSCT computer tomography apparatus 400, and a cross-sectional view.

In the case of the CSCT computer tomography apparatus 400, a distance between the X-ray tube 302 and the first detecting elements 304 differs from (according to the described embodiment: is smaller) a distance between the X-ray tube 302 and the second detecting elements 305. The first detecting elements 304 are provided in a first casing (not shown), and the second detecting elements 305 are provided in a second casing (not shown) which is provided separately from the first casing.

In a case that an energy-resolving detector 304 shall be retrofitted as an add-on to an already existing CT apparatus, or in a case in which the design changes related to an already existing CT scanner shall be kept small, the configuration according to FIG. 4 is advantageous. According to this configuration, the energy-resolving detector 304 is provided in a housing which is separate from a housing in which the non-energy-resolving detector 305 is provided. Further, the distance of the two detectors 304, 305 to the object of interest 301 may differ, as shown in FIG. 4. According to FIG. 4, this distance between the energy-resolving detector 304 and the X-ray tube 302 is shorter than the distance between the non-energy-resolving detector 305 and the X-ray tube 302. Alternatively, it can also be arranged at a larger distance.

Both detectors 304, 305 can be provided behind common shared focusing blades 308. However, alternatively to FIG. 4, separate collimators 308 can be used for the two detectors 304, 305. Also, constant lengths (see FIG. 3) or different lengths (see FIG. 4) of the blades 308 are possible.

FIG. 4 shows an embodiment with two separate detectors 304, 305. As an alternative to FIG. 4, different portions of the first detectors 304 can be arranged at both sides of the second detecting elements 305. The detector 304 can be provided at the same distance to the object of interest 301, or can be arranged closer to the object of interest 301, or at a larger distance.

In other words, in the embodiment shown in FIG. 4, the non-energy-resolving detector 305 is located around the fan-beam area 303. The energy-resolving detector 304 is a separate component, it may have a separate housing. Hence, a conventional CT could be upgraded with an energy-resolving detector 304. A common collimator 308 or two separate collimators 308 can be used for the energy-resolving detector 304 and for the non-energy-resolving detector 305.

In the following, referring to FIG. 5, a CSCT computer tomography apparatus 500 according to a third embodiment of the invention will be described.

According to the geometries described referring to FIG. 3 and FIG. 4, both detectors 304, 305 measure different angular regions. This could be changed in a configuration as shown in FIG. 5. According to FIG. 5, the primary beam 303 is shifted in such a manner that it does not impinge on a centre of the primary detector, but at a border portion. Consequently, in one direction of the scatter angles, a non-energy-resolved measuring is carried out, and in the other direction, an energy-resolved measurement is carried out.

Thus, FIG. 5 shows an asymmetric geometry for a combined energy-resolved/non-energy-resolved CSCT apparatus 500. This asymmetry can be obtained by shifting the detectors 304, 305, by shifting the X-ray tube 302 or by an adaptation/change of the slits formed by the blades 308. In other words, according to the embodiment shown in FIG. 5, the two detectors 304, 305 are arranged asymmetrically concerning the fan-beam 303.

In the following, referring to a flow diagram 600 shown in FIG. 6, an embodiment of the method of examining an object of interest with a computer tomography apparatus according to the invention will be described.

The method described referring to FIG. 6 includes emitting X-rays to an object of interest, namely a baggage item to be examined. Further, X-rays are detected which are coherently scattered from the object of interest in an energy-resolving manner. Moreover, X-rays coherently scattered from the baggage item of interest are detected in a non-energy-resolving manner. Beyond this, it is determined, based on non-energy-resolved detecting signals and, if desired, based on energy-resolved detecting signals, structural properties concerning the baggage item under inspection.

According to the method described referring to FIG. 6, complementary information obtained by using both methods (detecting energy-resolved as well as non-energy-resolved signals) can be used for the evaluation.

A multiple-level evaluation process may be carried out. An example for such an evaluation process is shown in FIG. 6. According to the fan-beam mode, CT projections and non-energy-resolved CSCT projections are measured. These data can be measured with the same detector. A subsequent automatic analysis of the CT image yields either the result that a suspicious region has been found, or the region is alternatively considered to be not dangerous. Only in the case that a region is considered to be suspicious, the non-energy-resolved CSCT data are reconstructed and evaluated. “Evaluation” means comparing the reconstructed scatter functions with information from a library database and an analysis of similarity. If the region is still considered to be suspicious after this analysis, energy-resolved CSCT projections are measured, reconstructed and evaluated according to a similar method. Alternatively or in addition, energy-resolved CSCT projection data measured during the CT-scan and/or the non-energy-resolved CSCT-scan may be used for reconstruction and evaluation. This results in a final statement concerning the allowance of the baggage item or the generation of an alarm.

The advantage of this method compared to the known realization of a CT and energy-resolved CSCT is, that energy-resolved CSCT does only have to be measured in few cases. Thus, measurement time is saved and the velocity of processing is increased.

Another use of the combined CSCT data according to another embodiment of the method of the invention could be that the reconstructed non-energy-resolved scatter functions can be used as a “convergence criterion” when reconstructing the energy-resolved scatter functions in the frame of an iterative reconstruction technique.

The non-energy-resolved scatter functions differ from the energy-resolved scatter functions in that they do not have such a good resolution in x-direction. This means that they can be gained from the energy-resolved scattering functions by convolution with a broad resolution function R. On the other hand, the non-energy-resolved scattering functions include less noise. Thus, the relatively poorly resolved scatter functions can be used as an envelope for the energy-resolved scatter functions. During this reconstruction, a further convergence criteria can be defined: “convergence is obtained when the degree of similarity A is minimum”. The degree of similarity A can be defined:

A=(F_{E}*R−F_{NE})² (2)

In equation (2), F_{E} und F_{NE} are the reconstructed energy-resolved (“E”) and non-energy-resolved (“NE”) scattering functions. A is the resolution function which broadens the energy-resolved scattering functions by a convolution in such a manner that the resolution now refers to a resolution of the non-energy-resolved resolution.

Alternatively, instead of the square in equation (1), another definition for the degree of similarity A (for example the absolute value) can be used.

Exemplary technical fields, in which the present invention may be applied advantageously, include baggage inspection, medical applications, material testing, and material science. An improved image quality and a reduced amount of calculations in combination with a low effort may be achieved. Also, the invention can be applied in the field of heart scanning to detect heart diseases.

1. A computer tomography apparatus (300) for examination of an object of interest (301), the computer tomography apparatus (300) comprising
an X-ray source (302) adapted to emit X-rays to an object of interest (301);
first detecting elements (304) adapted to detect X-rays coherently scattered from an object of interest (301) in an energy-resolving manner;
second detecting elements (305) adapted to detect X-rays coherently scattered from an object of interest (301) in an non-energy-resolving manner;
a determination unit (306) adapted to determine, based on detecting signals received from the first detecting elements (304) and/or from the second detecting elements (305), structural information concerning the object of interest (301).

2. The computer tomography apparatus (300) according to claim 1, being adapted as a coherent scatter computer tomography apparatus.

3. The computer tomography apparatus (300) according to claim 1, comprising a collimator (307) arranged between the X-ray source (302) and the first and second detecting elements (304, 305), the collimator (307) being adapted to collimate an X-ray beam emitted by the X-ray source (302) to form a fan-beam (303).

4. The computer tomography apparatus (300) according to claim 1, wherein the first detecting elements (304) and the second detecting elements (305) form a two-dimensional detector array.

5. The computer tomography apparatus (300) according to claim 1, wherein a distance between the X-ray source (302) and the first detecting elements (304) essentially equals a distance between the X-ray source (302) and the second detecting elements (305).

6. The computer tomography apparatus (300) according to claim 1, wherein a distance between the X-ray source (302) and the first detecting elements (304) differs from a distance between the X-ray source (302) and the second detecting elements (305).

7. The computer tomography apparatus (300) according to claim 1, wherein the first detecting elements (304) are adapted to detect X-rays scattered from an object of interest (301) into a first angle portion, and wherein the second detecting elements (305) are adapted to detect X-rays scattered from an object of interest (301) into a second angle portion.

8. The computer tomography apparatus (300) according to claim 7, wherein the first angle portion includes larger angles than the second angle portion.

9. The computer tomography apparatus (300) according to claim 8, wherein the first detecting elements (304) are divided into first sub-elements and into second sub-elements, wherein the second detecting elements (305) are arranged between the first sub-elements and the second sub-elements.

10. The computer tomography apparatus (300) according to claim 1, comprising one or more collimating blades (308) arranged on the first detecting elements (304) and/or on the second detecting elements (305) and being aligned to point towards the X-ray source (302).

11. The computer tomography apparatus (300) according to claim 1, wherein the first detecting elements (304) and the second detecting elements (305) are provided in a common casing.

12. The computer tomography apparatus (300) according to claim 1, wherein the first detecting elements (304) are provided in a first casing and the second detecting elements (305) are provided in a second casing which is different from the first casing.

13. The computer tomography apparatus (300) according to claim 1, wherein the X-ray source (302) is arranged with respect to the first detecting elements (304) and the second detecting elements (305) such that X-rays being transmitted through an object of interest (301) impinge on a non-central portion of the first detecting elements (304) and/or the second detecting elements (305).

14. The computer tomography apparatus (300) according to claim 1, wherein the first detecting elements (304) and the second detecting elements (305) are arranged on both sides of a primary fan-beam as emitted X-rays such that a measured angular range of the first detecting elements (304) and the second detecting elements (305) are essentially equal.

15. The computer tomography apparatus (300) according to claim 1, configured as one of the group consisting of a baggage inspection apparatus, a medical application apparatus, a material testing apparatus and a material science analysis apparatus.

16. A method of examining an object of interest (301) with a computer tomography apparatus (300),
the method comprising the steps of
emitting X-rays to an object of interest (301);
detecting X-rays coherently scattered from the object of interest (301) in an energy-resolving manner;
detecting X-rays coherently scattered from the object of interest (301) in a non-energy-resolving manner;
determining, based on energy-resolved detecting signals and/or on non-energy-resolved detecting signals, structural information concerning the object of interest (301).

17. The method according to claim 16,
further comprising the steps of
detecting X-rays transmitted through the object of interest (301);
determining, based on the detected transmitted X-rays; whether a further analysis is necessary.

18. The method according to claim 16,
further comprising the steps of
determining, based on non-energy-resolved detecting signals, without considering energy-resolved detecting signals, structural information concerning the object of interest (301);
analyzing the determined structural information, to decide whether a further determination is desired;
only if it is decided that a further determination is desired, determining, based on energy-resolved detecting signals, structural information concerning the object of interest (301).

19. The method according to claim 18,
wherein additional energy-resolved scatter data are acquired in a subsequent step.

20. The method according to claim 18,
wherein the decision whether a further determination is desired includes comparing structural information concerning the object of interest (301) determined based on non-energy-resolved detecting signals with data of a database.

21. The method according to claim 16,
wherein structural information concerning the object of interest (301) is determined based on energy-resolved detecting signals, wherein the non-energy-resolved detecting signals are used as a convergence criterion.
